(19) United States
(12) Patent Application Publication
(10) Pub. No.: US 2020/0069471 A1
(43) Pub. Date: Mar. 5, 2020

(54) GOGGLE MASK AND STRAP FIX STRUCTURE THEREOF

(71) Applicant: QBAS CO., LTD., Taipei (TW)

(72) Inventor: Chih-Cheng SHIUE, Taipei (TW)

(21) Appl. No.: 16/555,094

(22) Filed: Aug. 29, 2019

Related U.S. Application Data
(60) Provisional application No. 62/725,332, filed on Aug. 31, 2018.

(30) Foreign Application Priority Data
Mar. 19, 2019 (TW) 108109378

(51) Int. Cl. A61F 9/02; A44B 17/00

(52) U.S. Cl. CPC A61F 9/027; A63B 33/002; A44B 17/0023

(57) ABSTRACT

A strap fix structure for a goggle mask with a strap body and two side buckles is provided. The strap body includes a middle portion, two tail end portions and two buckle attachments. The tail end portions are disposed on two opposite sides of middle portion and the buckle attachments are disposed on distal ends of tail end portions respectively. The side buckles are disposed on two sides of a frame portion of the goggle mask respectively. The two tail end portions of the strap body are firstly passed through the two side buckles respectively and then folded up. The two buckle attachments disposed on the distal ends buckle folded parts of the two tail end portions to other parts of the two tail end portions respectively. The other parts are not passed through the side buckles.

### CROSS-REFERENCES TO RELATED APPLICATIONS

This application claims priority to U.S. Provisional Patent Application No. 62/725,332 filed on Aug. 31, 2018 and Taiwan Patent Application No. 108109378 filed on Mar. 19, 2019, which are hereby incorporated by reference in their entirety.

### BACKGROUND OF THE INVENTION

### Field of the Invention

The present invention provides a google mask and a strap fix structure. In particular, the strap fix structure for the goggle mask is used for underwater sports.

### Descriptions of the Related Art

Users often wear diving or swimming goggles while snorkeling, diving or swimming to ensure that the eyes of the users are protected and objects underwater can be seen clearly. For simplicity, the terms “diving goggles” and “swimming goggles” are referred to as goggle masks collectively hereinafter.

Current goggle masks have a strap that is fastened to the head of a user so that the goggle mask is fixed and fitted tightly around the eyes of the user. Generally, when the user adjusts the strap so that the goggle mask is worn comfortably with the proper tightness, the two tail end portions of the strap that are passed through the left and right sides of the goggle mask will have redundant parts of unequal lengths. The redundant parts will swing due to the movement of the head or the water flow if the redundant parts are not fixed, and this will cause interference or discomfort to the user (as shown in FIG. 1).

To solve the aforesaid problem, in the prior art, rings such as D ring or tri-glide buckles are respectively provided at the two tail end portions of the strap to tie and fasten the redundant parts. However, the additional rings or buckles cause the following problems when the goggle mask is worn: (1) the rings or buckles are made of a relatively hard material, so the user wearing the goggle mask will feel uncomfortable where the rings/buckles come into contact with the skin thereof; (2) the adjustment of the rings/buckles is labor-consuming due to the relatively large resistance or friction between the rings/buckles and the strap; (3) during the adjustment of the length of the strap, the rings/buckles rub the skin or get stuck to the hair or the swimming cap of the user and cause discomfort; (4) the rings/buckles need to be mounted on the strap manually, so additional manpower is required during the manufacturing process.

U.S. Pat. Nos. 5,046,200, 6,349,421B2 and 6,256,847B1 have respectively disclosed the use of velcro, snap fasteners, and the use of projecting parts on both sides of the lens frame to pass through the corresponding openings or continuous teeth in the strap to further fix the strap such as the head strap/the watch strap or the like. However, the application of the aforesaid prior art to the goggle mask may still cause many problems, such as getting stuck to the hair, inflexible adjustment due to the fixed distance of the snap holes, dependence on finger sensation to achieve alignment and attachment after wearing the goggle mask, or a complicated manufacturing process or the like.

On the other hand, some manufacturers directly discard the use of the rings and instead design the buckling end of the strap that is passed through the goggle mask to be longer or bent inwardly so that the end portion of the strap is less likely to swing. However, when the goggle mask is manufactured into a finished product and packaged before leaving the factory, such a practice requires a large amount of manpower to neaten the strap to maintain concise packaging and while ensuring good impression to the consumer purchasing the product, thereby, consuming a lot of time and manpower.

Accordingly, it is important in the art to prevent the swing of the strap of the goggle mask while underwater and eliminating the discomfort and inconvenience with the buckles (called buckle attachment hereinafter to avoid confusion with side buckles) that are currently available.

### SUMMARY OF THE INVENTION

An objective of the present invention is to provide a goggle and a strap fix structure for the goggle mask, which can be used by a user for fixing the tail ends of the strap, thereby, preventing the swing of the redundant parts of the strap after being worn due to the movement. At the same time, the strap fix structure is comfortable, easily adjustable and convenient to package. To achieve the aforesaid objective, the strap fix structure of the present invention comprises a strap body and two side buckles. The strap body comprises a middle portion, two tail end portions disposed on two opposite sides of the middle portion, and two buckle attachments disposed on the distal ends of the tail end portions respectively. The two side buckles are disposed on the two sides of a frame portion of the goggle mask respectively. The two tail end portions of the strap body are firstly passed through the two side buckles respectively and then folded up. The two buckle attachments disposed on the distal ends buckle folded parts of the two tail end portions to other parts of the two tail end portions respectively, i.e. the folded parts of the two tail end portions are buckled to other parts of the two tail end portions via two buckle attachments disposed on the distal ends of the folded parts. The other parts are not passed through the side buckles.

To achieve the aforesaid objective, the inside surface of each of the two side buckles included in the strap fix structure of the present invention comprises a fixing element. The fixing element has a height decreasing from a direction away from the goggle mask to a direction close to the goggle mask.

To achieve the aforesaid objective, the fixing element included in the strap fix structure of the present invention is formed integrally with the side buckle and is formed as ribs, strips or bumps.

To achieve the aforesaid objective, an arm portion included in the strap fix structure of the present invention has a width which is 5 millimeters or ranges from 8 millimeters to 20 millimeters.

To achieve the aforesaid objective, the arm portion included in the strap fix structure of the present invention has a length which is ranges from one-fourth times the width of the tail end portion to two times the width of the tail end portion.

To achieve the aforesaid objective, the buckle attachment included in the strap fix structure of the present invention comprises two sub-arm portions which extend towards each other and define a gap therebetween. The other part of the tail end portion of the strap body that is not passed through the side buckle is buckled by passing through the gap.

To achieve the aforesaid objective, the front shape of the two sub-arm portions included in the strap fix structure of the present invention is a triangle, a circle, a square, a ellipse, or a polygon. The cross section of the two sub-arm portions is M-shaped, W-shaped or C-shaped.

To achieve the aforesaid objective, the gap defined by the two sub-arm portions included in the strap fix structure of the present invention is sealed by a rubber, adhesive or sealing element.

To achieve the aforesaid objective, the buckle attachments included in the strap fix structure of the present invention comprises a fastener and a band. The band surrounds the other part of the tail end portion that is not passed through the side buckle in a top-down or bottom-up manner and then is hitched to the fastener so that the folded part of the tail end portion is buckled to other part of the tail end portion that is not passed through the side buckle.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a schematic view illustrating an unfixed strap of a conventional goggle mask swings;

FIG. 2A to FIG. 2B are schematic views illustrating a strap fix structure of the present invention that is fixed;

FIG. 3 is a schematic view illustrating the strap body of the strap fix structure of the present invention that is spread out;

FIG. 4 is a schematic view illustrating the side buckle of the strap fix structure of the present invention comprising the fixing element;

FIG. 5 is a schematic cross-sectional view illustrating the strap body of the strap fix structure of the present invention passing through the side buckle and then is folded up;

FIG. 6A is a schematic view illustrating the first embodiment of a buckle attachment comprised in the strap body of the strap fix structure according to the present invention;

FIG. 6B is a schematic view illustrating the second embodiment of a buckle attachment comprised in the strap body of the strap fix structure according to the present invention;

FIG. 6C is a schematic view illustrating the strap fix structure of FIG. 6B in a fixed state;

FIG. 7A to FIG. 7D are schematic views respectively illustrating various aspects of the buckle attachment included in the strap fix structure of the present invention when observed from the front side;

FIG. 8A to FIG. 8D are schematic views respectively illustrating different cross-sectional shapes of the buckle attachment included in the strap fix structure of the present invention;

FIG. 9A to FIG. 9B are schematic views illustrating the step of sealing the buckle attachment comprised in the strap fix structure according to the second embodiment of the present invention via post-processing; and

FIG. 10 is a schematic view illustrating the third embodiment of a buckle comprised in the strap body of the strap fix structure according to the present invention.

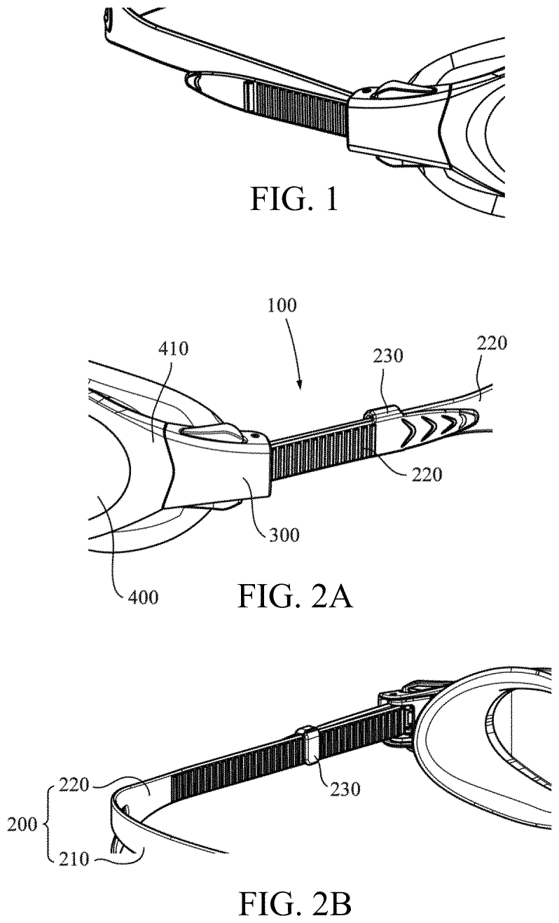

### DESCRIPTION OF THE PREFERRED EMBODIMENT

According to embodiments of the present invention, a strap fix structure 100 for a goggle mask 400 and the google mask comprising the strap fix structure 100 are disclosed; the strap fix structure 100 comprises a strap body 200, and the google mask 400 comprises a frame portion 410, two side buckles 300 and the strap fix structure 100. The contents of the elements are described as follows.

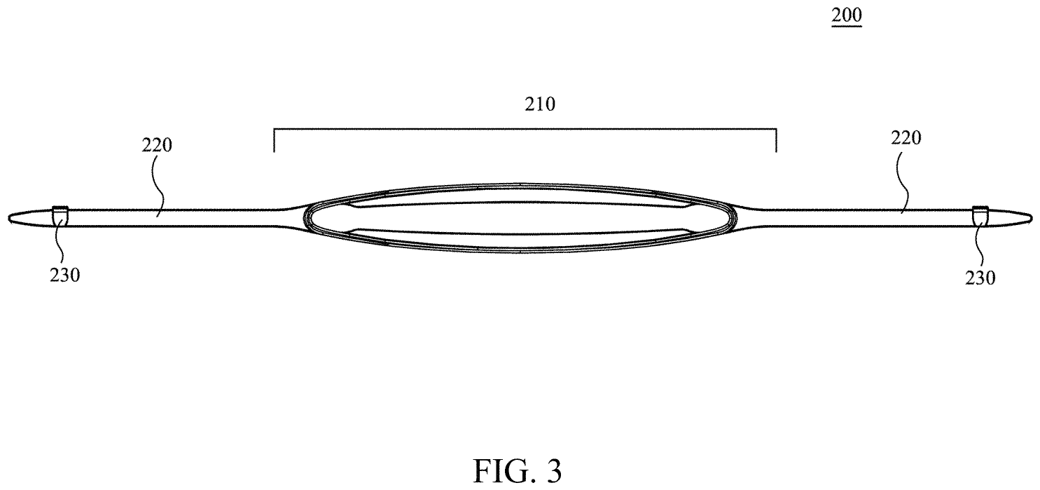

In detail, as shown in FIG. 2A, FIG. 2B and FIG. 3, the strap body 200 comprises a middle portion 210, two tail end portions 220 disposed on two opposite sides of the middle portion 210, and two buckle attachments 230 disposed on distal ends of the tail end portions 220 respectively. The two side buckles 300 are disposed on two sides of the frame portion 410 of the goggle mask 400 respectively (the frame portion 410 is provided with a lens therein). The two tail end portions 220 of the strap body 200 are firstly passed through the two side buckles 300 respectively and then folded up; namely, the tail end portion 220 is threaded through the side buckles 300 to form a first part and a second part which are divided by the side buckles 300; the second part is between the middle portion 210 and the side buckle 300 and is not yet passed through the side buckle 300, while the first part is passed through the side buckle 300; the lengths of the first and the second parts can be adjusted. When the user wears the google mask 400, the second part of the tail end portion 220 is in contact with the user's head, while the first part is not. The first part is beside the second part along the width direction of the google mask 400, and may swing due to the movement of the head or the water flow if not fixed or hold by the buckle attachment 230.

Specifically, the buckle attachment 230 is disposed on the distal end (i.e. the first part), and can buckle up the first part and the second part. In other words, the tail end portions 220 that are threaded through the side buckles 300 and then folded up are adapted to be buckled by the buckle attachments 230. Thus, the first part of the tail end portions 220 cannot swing from the second part.

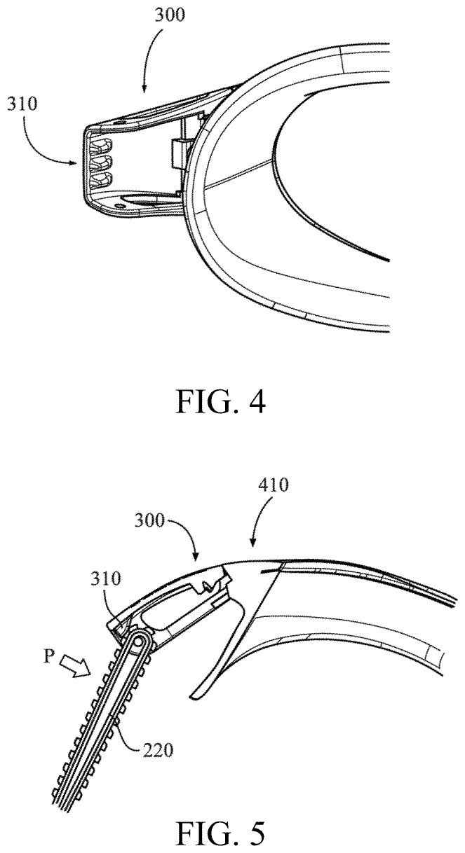

As shown in FIG. 4, each of the two side buckles 300 may be provided with a fixing element 310. The fixing element 310 is disposed at the inside surface of the side buckle 300. The fixing element 310 may be one or more protrusions, and formed as ribs, strips or bumps (aspects of the latter two forms are not shown in the figure). The fixing element 310 may further be formed integrally with the side buckle 300.

In detail, the fixing element 310 protrudes inwardly from the inside surface. The protruding height thereof decreases from the outside to the inside (i.e., from a direction away from the goggle mask to a direction close to the goggle mask) so that, as shown in FIG. 5, a downward pressure P may be imposed on the tail end portion 220 that is attached to the fixing element 310 after being passed through the side buckle 300. The downward pressure P makes the first part of the tail end portion 220slant towards the other (second) part of the tail end portion 220. As a result, the position of the folded tail end portion 220 is less likely to upwarp or shake during use.

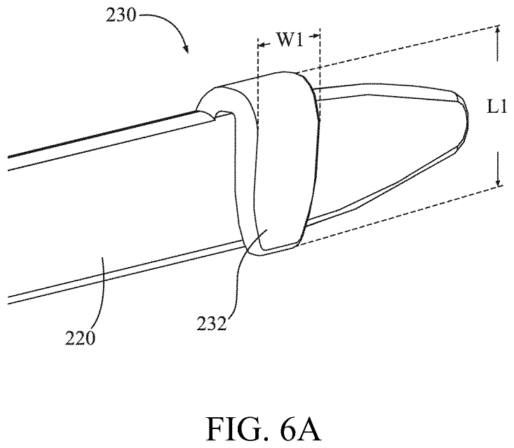

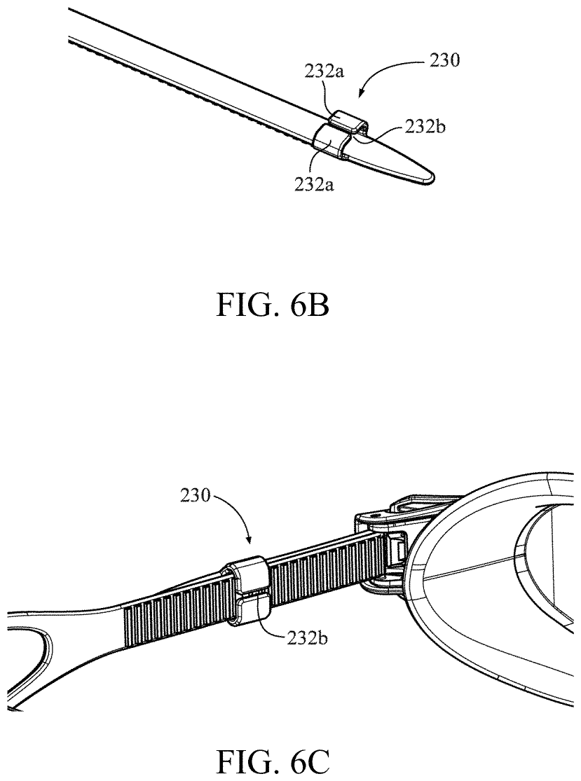

Moreover, with reference to FIG. 6A, the strap body 200 fixes the tail end portion 220 that is folded up after being passed through the side buckle 300 via the arrangement of the buckle attachment 230. In detail, in the first embodiment shown in FIG. 6A, the buckle attachment 230 may comprise an arm portion 232. The arm portion 232 extends from an upper side or lower side of the tail end portion 220 towards another lower side or upper side of the tail end portion 220, so the arm portion 232 may have a hook shape with a downward or upward opening (a cross section thereof is similar to an upright or inverted L shape). The arm portion 232 may be buckled to the second part of the tail end portion 220 in a top-down or bottom-up manner depending on the habits of the user.

In other words, by sandwiching the arm portion 232 between the second part of the tail end portion 220 and the head of the user, the folded tail end portion 220 can be attached more closely to the head of the user so that it will not swing around and cause interference to the user during the underwater sport.

With reference to FIG. 6A, in this embodiment, the arm portion 232 has a length L1 and a width W1. When the value of the length L1 of the arm portion 232 is larger than or equal to a width W2 of the tail end portion 220 of the strap body 200, the arm portion 232 has sufficient friction to assist in the buckling of the buckle attachment 230. When the value of the length L1 of the arm portion 232 is smaller than the width W2 of the tail end portion 220 of the strap body 200 (e.g., in a range from one-fourth times the width W2 of the tail end portion 220 to two times the width W2 of the tail end portion 220), the width W1 of the arm portion 232 is preferably 5 millimeters or ranges from 8 millimeters to 20 millimeters so that it can be touched by the fingers of the user and meanwhile provide sufficient friction to assist in the buckling of the buckle attachment 230.

Additionally, although the arm portion 232 shown in FIG. 6A is formed by extending with the same width, it is not limited thereto. In other words, the length L1 and the width W1 of the arm portion 232 may also be changed in other manners to provide a sufficient fastening force.

In the second embodiment of the buckle attachment 230 as shown in FIG. 6B, the buckle attachment 230 may have two sub-arm portions 232a. The two sub-arm portions 232a have hook shapes respectively with a downward opening and an upward opening and are disposed opposite to each other. That is, the two sub-arm portions 232a are extended towards each other respectively from an upper side and lower side of the tail end portion 220 towards another lower side and upper side of the tail end portion 220. The two sub-arm portions 232a do not contact with each other, thereby, having a cross section that is similar to an upright or inverted L shape. In this way, the two sub-arm portions 232a will define a gap 232b therebetween after being extended towards each other. The gap 232b may be changed due to an external force (e.g., a forced imposed by a hand for opening, squeezing or pinching) applied to the two sub-arm portions 232a to make it convenient for the user to buckle the first part of the tail end portion 220 to the second part of the tail end portion 220. Furthermore, the second part of the tail end portion 220 may also be directly hitched within the gap 232b without imposing a pressure to the gap 232b, thereby achieving the effect of buckling the folded tail end portion 220 to the second part of the tail end portion 220.

In other words, as shown in FIG. 6C, by sandwiching the second parts of the tail end portions 220 between both of the two sub-arm portions 232a, the folded tail end portion 220 can be attached more closely to the head of the user so that it will not swing around and cause interference to the user during the underwater sport.

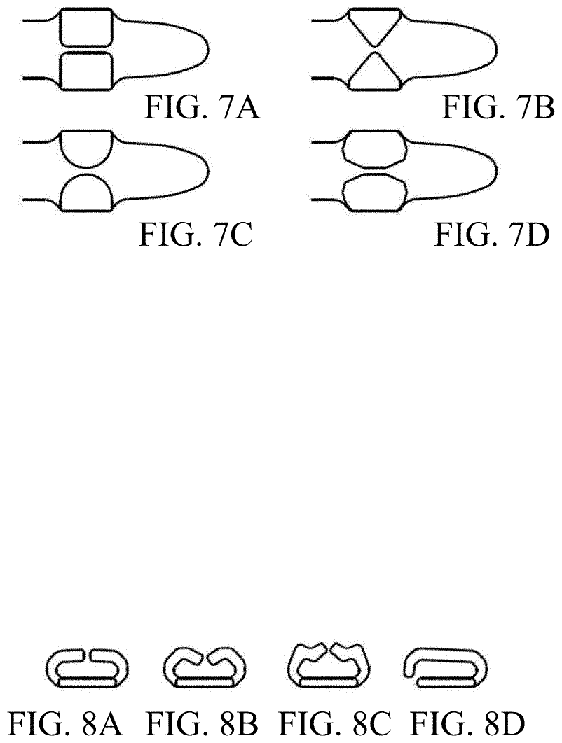

When observed from the front side, the appearance of the arm portion 232 of the aforesaid first embodiment and the two sub-arm portions 232a of the second embodiment is not limited to a rectangle, but may also be a triangle, a circle, a square, a ellipse, or a polygon or the like (as shown in FIG. 7A to FIG. 7D). Across section thereof may further be similar to a C shape, M shape, W shape or D shape or the like (as shown in FIG. 8A to FIG. 8D). The length and the width of the two sub-arm portions 232a of the second embodiment may also be changed in various manners to provide a sufficient fastening force.

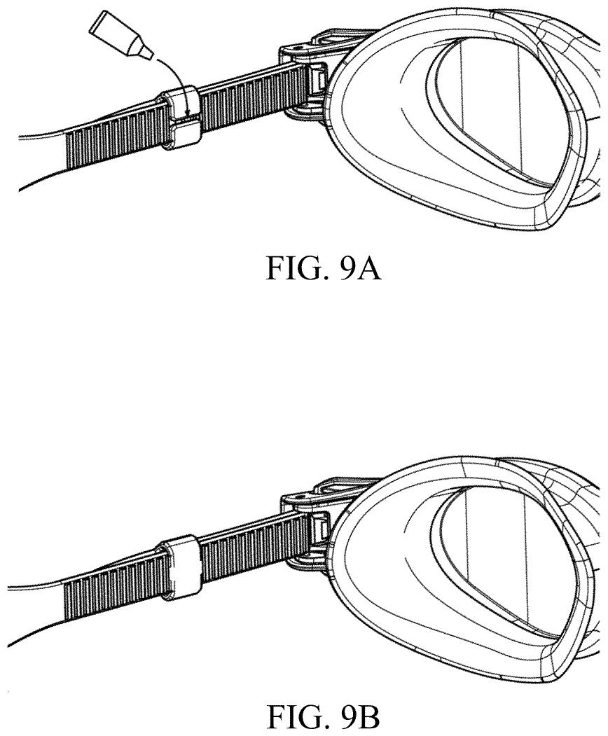

Additionally, FIG. 9A to FIG. 9B are schematic views illustrating a step of sealing the buckle attachment 230 according to the second embodiment of the present invention via post-processing. As shown in the figures, the two sub-arm portions 232a of the second embodiment may further seal the gap 232b via a step of post-processing, e.g., by rubberizing, welding or the like, so that the folded tail end portion 220 that is buckled is less likely to be released due to intense movement or an external force imposed accidentally.

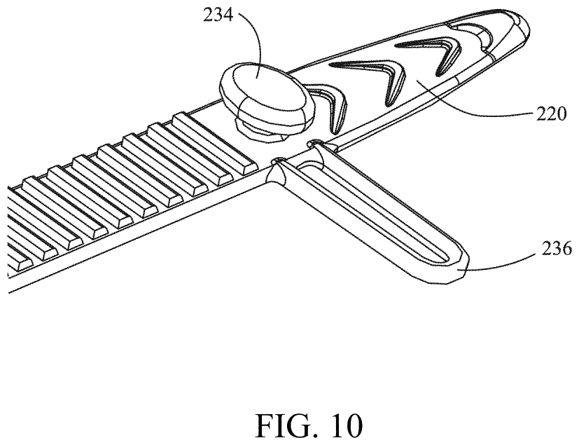

With reference to FIG. 10, the third embodiment of the buckle attachment 230 of the present invention may comprise a fastener 234 and a band 236. The fastener 234 protrudes from an external surface of the tail end portion 220 towards the direction away from the strap. The band 236 is an enclosed band and is disposed at the edge of the upper end or lower end of the tail end portion 220 corresponding to the position of the fastener 234. The band 236 has a certain length. Accordingly, when the user wears the goggle mask 400, by bending the band 236 in the direction towards the head of the user, the band 236 surrounds the second part of the tail end portion 220 and then is hitched to the fastener 234, thereby achieving the effect of fixing the position of the folded tail end portion 220 and fixing the folded tail end portion 220 (FIG. 10 only shows the state before the hitching).

With the aforesaid arrangement, after the user wears the goggle mask 400 on the head and the tail end portion 220 of the strap body 200 is passed through the side buckle 300, the folded tail end portion 220 is less likely to upwarp due to the downward pressure of the fixing element 310. The folded tail end portion 220 is fixed by the buckle attachment 230 so that the folded tail end portion 220 will not swing due to the water flow and thus cause interference.

Additionally, when the goggle mask provided with the strap fix structure of the present invention is manufactured into a finished product, the product can be packaged by an automatic production process without the need of additional manpower to perform the operation of providing the strap with the ring/buckle attachment, thereby, saving manpower and time and improving production efficiency. The buckle attachment of the present invention may further buckle the folded tail end portion after the strap is adjusted at the proper tightness, thereby avoiding or reducing the resistance in the adjustment. More particularly, the buckle attachment of the first embodiment and the second embodiment can reduce the resistance in the adjustment remarkably even when the folded tail end portion is buckled, because those are unenclosed.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A goggle mask, comprising:
a frame portion;
a lens, disposed in the frame portion;
two side buckles, disposed on two opposite sides of the frame portion respectively; and
a strap fix structure, comprising a strap body which includes a middle portion, two tail end portions disposed on two opposite sides of the middle portion, and two buckle attachments disposed on distal ends of the tail end portions respectively;
wherein the tail end portion is threaded through the side buckle of the goggle mask to form a first part and a second part which are divided by the side buckle, and the buckle attachment is disposed on the first part to buckle the first part and the second part.

2. The goggle mask according to claim 1, wherein an inside surface of each of the two side buckles comprises a fixing element, the fixing element has a height decreasing from a direction away from the goggle mask to a direction close to the goggle mask.

3. The goggle mask according to claim 2, wherein the fixing element is formed integrally with the side buckle and is formed as ribs, strips or bumps.

4. A strap fix structure for a goggle mask which comprises a frame portion a lens disposed in the frame portion and two side buckles disposed on two opposite sides of the frame portion respectively, the strap fix structure comprising;
a strap body, comprising a middle portion, two tail end portions disposed on two opposite sides of the middle portion, and two buckle attachments disposed on distal ends of the tail end portions respectively; and
wherein the tail end portion is threaded through the side buckle of the goggle mask to form a first part and a second part which are divided by the side buckle, and the buckle attachment is disposed on the first part to buckle the first part and the second part.

5. The strap fix structure according to claim 4, wherein an inside surface of each of the two side buckles comprises a fixing element, the fixing element has a height decreasing from a direction away from the goggle mask to a direction close to the goggle mask.

6. The strap fix structure according to claim 5, wherein the fixing element is formed integrally with the side buckle and is formed as ribs, strips or bumps.

7. The strap fix structure according to claim 4, wherein the buckle attachment comprises an arm portion which is buckled to the second part of the tail end portion in a top-down or bottom-up manner.

8. The strap fix structure according to claim 7, wherein the arm portion has a width which is 5 millimeters or ranged from 8 millimeters to 20 millimeters.

9. The strap fix structure according to claim 8, wherein the arm portion has a length which is in a range from one-fourth times a width of the tail end portion to two times the width of the tail end portion.

10. The strap fix structure according to claim 4, wherein the buckle attachment comprises two sub-arm portions which are extended towards each other and define a gap therebetween, and the second part of the tail end portion is held by the sub-arm portions after passing through the gap.

11. The strap fix structure according to claim 10, wherein a front shape of the two sub-arm portions is a triangle, a circle, a square, a ellipse, or a polygon, and a cross section of the sub-arm portions is M-shaped, W-shaped or C-shaped.

12. The strap fix structure according to claim 11, wherein the gap between the two sub-arm portions is sealed by rubber, adhesive or sealing element.

13. The strap fix structure according to claim 4, wherein the buckle attachment comprises a fastener and a band, and the band surrounds the second part of the tail end portion in a top-down or bottom-up manner and then is hitched to the fastener.
